(19) United States
(12) Patent Application Publication
(10) Pub. No.: US 2026/0182891 A1
(43) Pub. Date: Jul. 2, 2026

(54) SUBCUTANEOUS MULTICHANNEL WIRELESS ELECTROENCEPHALOGRAPHY SYSTEM FOR CHRONIC HOME USE

(71) Applicants: Brown University, Providence, RI (US); The General Hospital Corporation, Boston, MA (US)

(72) Inventors: Arto NURMIKKO, Providence, RI (US); Jihun LEE, Providence, RI (US); Ah-Hyoung LEE, Providence, RI (US); Sydney S. CASH, Cambridge, MA (US)

(21) Appl. No.: 19/126,620

(22) PCT Filed: Nov. 2, 2023
(86) PCT No.: PCT/US2023/078505

§ 371 Date: May 1, 2025

Related U.S. Application Data
(60) Provisional application No. 63/382,265, filed on Nov. 3, 2022.
(60) Provisional application No. 63/513,188, filed on Jul. 12, 2023.

(51) Int. Cl. A61B 5/293; A61B 5/00; A61B 5/31

(52) U.S. Cl. CPC A61B 5/293; A61B 5/0006; A61B 5/0031; A61B 5/31; A61B 5/6814; A61B 5/7225

(57) ABSTRACT

A system comprises one or more sensor devices for subcutaneous electroencephalography where each sensor device includes a flexible biocompatible polymer sensor strip adapted to be inserted into a cranial subdermal space and including a plurality of electrodes. The strip includes a microchip responsive to each of the electrodes and comprising a multi-channel integrated circuit system-on-chip with a wireless transceiver, a low-noise neural amplifier, a unique network address, and RF energy harvesting microcircuits. In one embodiment, the sensor strip is no more than about 1 mm wide, at least about 5 cm long, and no more than about 300 μm thick. An external transceiver is disposed on the exterior of the subject's skin near one or more sensor devices in secure wireless communication with the respective wireless transceivers comprised in the sensor devices microchips. The system is configured to communicate using a network communication protocol, such as time division multiple access or code division multiple access.

### CROSS-REFERENCE TO RELATED APPLICATIONS

This application claims the benefit of priority from U.S. Provisional Application No. 63/382,265 filed Nov. 3, 2022, and U.S. Provisional Application No. 63/513,188 filed Jul. 12, 2023, each of which is incorporated by reference in its entirety.

### BACKGROUND

### Field

The present disclosure relates generally to electroencephalography (“EEG”), and, in particular, to chronic EEG, and, further, to a system and method for subcutaneous EEG.

### Description of the Problem and Related Art

Currently, the principal diagnostic method for the large patient population suffering from intermittent seizures is based on short term stays in a hospital. Yet a clinically informative assessment of the disease could benefit fundamentally from recording brain activity continuously for many months. Accurate, reliable monitoring of brain activity over many months while epilepsy patients carry out everyday activities is not presently possible. If such a technology existed, it could provide valuable, clinically critical neurological diagnostic information for clinicians to decide on therapeutic strategies. By contrast, it is routine to have cardiac patients implanted with devices to record heart activity for weeks or even months in order to diagnose various arrhythmias and related adverse events.

Currently, making a diagnostic assessment such as for the location or type of seizure is based on short recordings made at the clinic over days. The limited time window is often incomplete and ambiguous in following the course of the disease, for making choices for effectiveness of medication therapies. The most common non-invasive technique uses scalp EEG which, while standard in the clinic, is not practical for 24/7 use over many months.

Recently, subcutaneous EEG has emerged as a possible approach to long-term epilepsy monitoring, offering in principle a number of advantages over scalp EEG systems including more reliable recording and reduction of electrical artifacts. This, however, assumes that an implant is minimally invasive, unobtrusive, wireless, and does not subtract from the already complex issues in the quality of life of a patient.

In addition to a number of wireless scalp EEG devices which have many practical limitations, there are currently two early clinical research efforts to develop wireless sub-scalp EEG devices, for the purpose of demonstrating chronic capability. One is a device being developed in Denmark including first clinical trials Denmark. See Weisdorf S, Duun-Henriksen J, Kjeldsen M J, Poulsen F R, Gangstad S W, Kjaer T W. Ultralong-term subcutaneous home monitoring of epilepsy—490 days of EEG from nine patients. Epilepsia. 2019 November; 60(11):2204-22!4. doi: 10.llll/epi.16360. The second is an effort mounted in Australia. See Benovitski Y B, Lai A, McGowan C C, Burns 0, Maxim V, Nayagam D A X, Niillard R, Rathbone G D, le Chevoir M A, Williams R A, Grayden D B, May C N, Murphy M, D'Souza \V J, Cook N I J, Williams C E. Ring and peg electrodes for minimally invasive and long-term sub-scalp EEG recordings. Epilepsy Res. 2017 September; 135:29-37. doi: 10.1016/j.eplepsyres.2017.06.003.

Each of these systems is based on a ˜10 cm long subcutaneous electrode strip which is inserted from behind the ear. The leads from the strip are connected to a commercial electronic amplifier/radio which locates subcutaneously behind the ear. The amplifier/radio form a module separate from the sensor strip and space for the module must be made by additional surgical intervention such as by creating a pour into tissue behind the ear for this for the additional step in the implant procedure.

Moreover, the sensors have only one or two sensing electrodes, and are not miniaturized. As such, the technology is not naturally scalable to larger channel counts or multiple strips and typically use commercial wireless telemetry modules. At the same time, in case of the Danish device for example, a limited number of human subjects have used the implant up to one year and beyond, results indicating a signal-to-noise ratio in the EEG recordings as comparable to from a single scalp electrode. See Weisdorf, et al.

### SUMMARY

For purposes of summary, certain aspects, advantages, and novel features of the system and method are described herein. It is to be understood that not necessarily all such advantages may be achieved in accordance with any one particular embodiment. Thus, the apparatuses or methods claimed may be embodied or carried out in a manner that achieves or optimizes one advantage or group of advantages as taught herein without necessarily achieving other advantages as may be taught or suggested herein.

In one aspect, the system comprises a sensor device for subcutaneous electroencephalography with a flexible biocompatible polymer sensor strip adapted to be inserted into a cranial subdermal space and including a plurality of electrodes. The strip includes a microchip responsive to each of the electrodes and comprising a multi-channel integrated circuit system-on-chip with a wireless transceiver, a low-noise neural amplifier, a unique network address, and RF energy harvesting microcircuits. In one embodiment, the sensor strip is no more than about 1 mm wide, at least about 5 cm long, and no more than about 300 μm thick. In another embodiment, the microchip includes a multi-turn on-chip antenna coil for coupling external RF energy to the RF energy harvesting circuitry. Typically, the electrodes are responsive to brain activity exhibiting frequencies between about 0.1 Hz to about 100 Hz.

In one aspect, the system also includes an external transceiver disposed on the exterior of the subject's skin near one or more sensor devices in secure wireless communication with the respective wireless transceivers comprised in the sensor devices microchips. The external transceiver may be configured to demodulate signals from multiple sensor devices. Advantageously, the system may employ a network communication protocol, such as a time division multiple access protocol.

According to a further aspect, one method disclosed herein includes inserting into a patient's cranial subdermal space, a biocompatible subcutaneous sensor that includes a flexible sensor strip that includes a plurality of electrodes and a microchip responsive to the electrodes, and placing an external transceiver on the exterior of the subject's skin in close proximity to the sensor. The microchip includes a multi-channel integrated circuit system-on-chip with a low-noise neural amplifier, data encoding wireless telemetry circuitry, RF energy harvesting microcircuits, and a wireless transceiver configured to transmit signals representative of brain activity using modulated backscatter RF energy, and where the external transceiver is in secure wireless communication with the wireless transceiver and configured to demodulate said signals.

A related method includes generating analog signals representative of brain activity with a biocompatible sensor strip inserted into a subject's cranial subdermal space, where such a sensor strip comprises a plurality of electrodes and a microchip adapted to be responsive to the plurality of electrodes, where the microchip includes a multi-channel integrated circuit system-on-chip with a low-noise neural amplifier, analog-to-digital circuits, a unique network address, RF energy harvesting microcircuits, and a wireless transceiver. The method further includes converting the signals from analog to digital signals and wirelessly transmitting the digital signals to a wireless transceiver located on the exterior of the subject's skin using modulated RF backscatter energy.

Further, the method may include the step of formatting the digital signals according to a shared medium network protocol prior to transmitting them to the wireless transceiver.

### BRIEF DESCRIPTION OF THE DRAWINGS

The system and related method are described with reference to the accompanying drawings. In the drawings, like reference numbers indicate identical or functionally similar elements. Additionally, the left-most digit(s) of a reference number identifies the drawing in which the reference number first appears.

FIG. 1 is a diagram of an exemplary embodiment of the subcutaneous wireless EEG system;

FIG. 2 illustrates an exemplary embodiment of a sensor strip for use in the system shown in FIG. 1;

FIG. 3 is a broken side view of an exemplary sensor strip;

FIG. 4 is a functional schematic of the exemplary system of FIG. 1;

FIG. 5 is a detailed schematic of an exemplary sensor strip for use the system of FIG. 1;

FIG. 6 is a schematic diagram of an exemplary two-stage, low noise, neural amplifier that may be implemented in the system of FIG. 1; and

FIG. 7 is a flowchart depicting an exemplary method that may be performed by the system.

### DETAILED DESCRIPTION

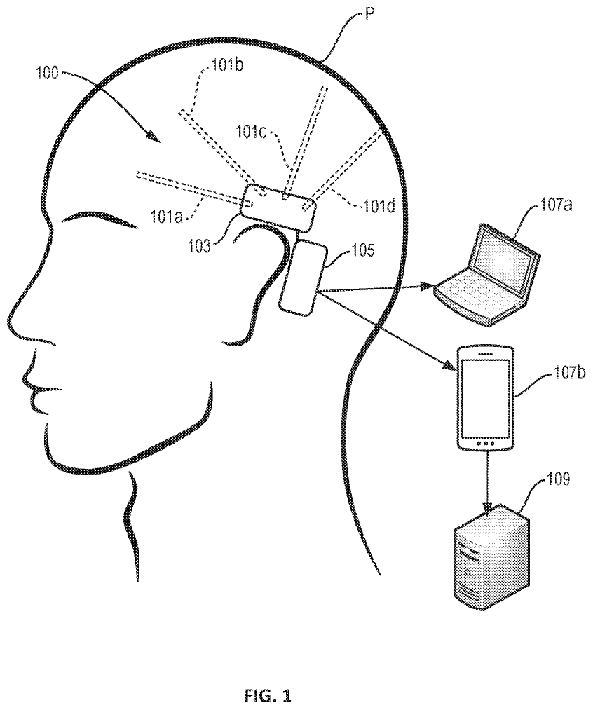

The various embodiments of the system and their advantages are best understood by referring to FIGS. 1 through 7 of the drawings. The elements of the drawings are not necessarily to scale, emphasis instead being placed upon clearly illustrating the novel features and principles of operation. Throughout the drawings, like numerals are used for like and corresponding parts of the various drawings.

Furthermore, reference in the specification to “an embodiment,” “one embodiment,” “various embodiments,” or any variant thereof means that a particular feature or aspect described in conjunction with the particular embodiment is included in at least one embodiment. Thus, the appearance of the phrases “in one embodiment,” “in another embodiment,” or variations thereof in various places throughout the specification are not necessarily all referring to its respective embodiment.

FIG. 1 depicts an exemplary arrangement of a system 100 for chronic EEG disposed proximally to the temporal lobe of patient P. One or more sensor strips 101a-d are implanted subcutaneously between the epidermis and the cranium of patient P and are implanted in a fan arrangement such that near end(s) of strip(s) 101a-d are located near epidermal coil antenna 103. Antenna 103 is coupled to epidermal EEG hub device 105 which is situated on the skin behind patient's P ear and may be affixed to the patient's scalp with a suitable adhesive. EEG hub device 105 is in wireless communication with computer-based devices 107a, b. Devices 107a, b may be in communication with a computer-based server 109, which may be a cloud server.

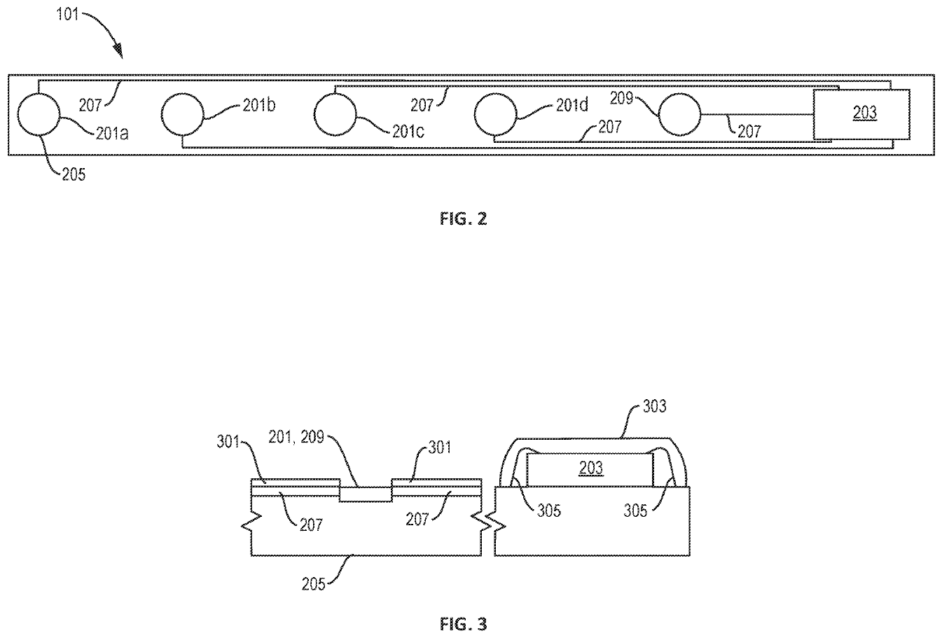

FIG. 2 is a diagram of sensor strip 101 which comprises a biocompatible, flexible polyimide substrate 205 supporting a plurality of recording electrodes 201a-d that are coupled via leads 207 to microchip 203 described in greater detail below. Strip 101 may also comprise a reference electrode 209, also coupled to microchip 203. Microchip 203 can be a system-on-chip (“SoC”) or an application-specific integrated circuit (“ASIC”), preferably no more than about 800 μm by about 500 μm in area and no more than about 300 μm in thickness. Strip 101 is at least about 5 cm in length and up to about 10 cm or more depending upon the geometry of the cranium. Strip 101 is preferably no more than about 1 mm wide and no more than about 300 μm thick. These dimensions are achieved through integrating the microchip 203, and the electrodes 201a-d on the single polyimide substrate 205. Microchip 203 and electrodes 201a-d are assembled as a single, seamless unit or structure with the polyimide substrate 205 such as by using wire-bonding or flip-chip bonding technique. Such minimally invasive strips which house the sensing and radio-frequency transmission functions in one minimally obtrusive subdermal unit can be injected under the scalp e.g., using an appropriately gauged hypodermic needle or a custom catheter.

Further, this novel arrangement suggests modularity whereby multiple multielectrode EEG sensor strips 101 can be combined in a web of unobtrusive implants to cover a large brain area (e.g., the temporal lobe). Each sensor strip 101 detects neural signals from the set of electrodes 201a-d along the strip which are relayed to the dedicated microchip 203. Microchip 203 is custom designed and able to record microvolt-size EEG signals simultaneously from several electrodes 201a-d by multiplexing techniques, the electronics operating in common-mode noise suppression geometry as discussed in greater detail below.

FIG. 3 is a broken, side view of an exemplary sensor strip 101 showing placement of microchip 203 and electrodes 201, 209 with respect to polyimide substrate 205. Electrodes 201, 209 are encapsulated with a polymeric material 301, for example, liquid crystal polymer, polyimide and/or parylene because of its biocompatibility, flexibility, and relatively good thermal/chemical stability. Microchip 203 is affixed to strip 205 with wire bonding 305 or flip chip bonding and encapsulated with thin-layer packaging e.g., using dioxide layers by atomic-layer deposition (ALD) or parylene.

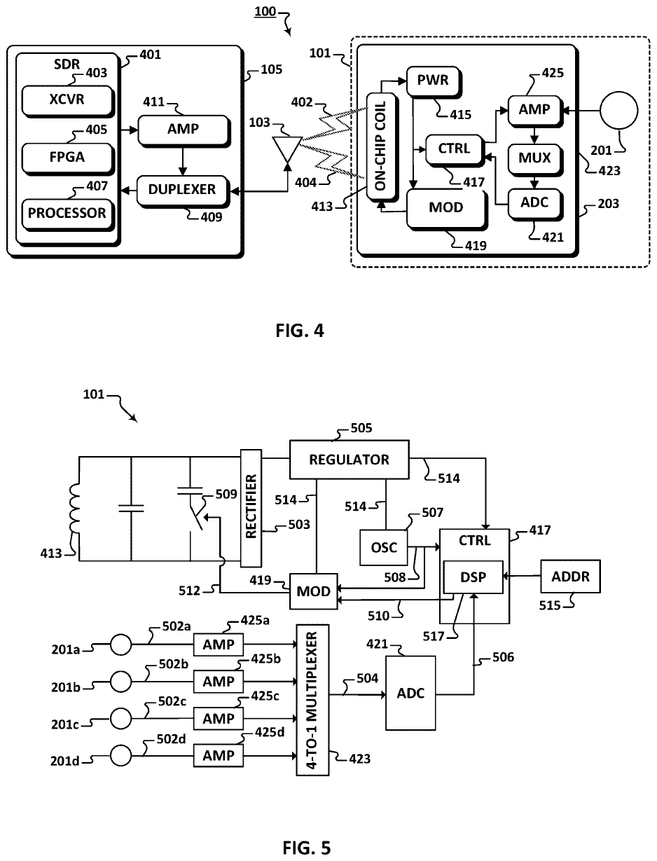

Referring to FIG. 4, microchip 203 comprises power management module 415 responsive to on-chip inductive coil 413. Coil 413 is inductively coupled to EEG hub device 105 via epidermal antenna 103. Microchip 203 further comprises a control module 417 that can be a processor. Control module 417 may include a digital signal processor (FIG. 5: 517) for processing digitized EEG signals according to the methods disclosed herein, and may include a memory, such as, without limitation, read-only memory (ROM), or random access memory (RAM), such as SRAM, DRAM, or non-volatile memory for data storage. Digital signal processor 517 may further include signal filtering such as, without limitation, bandpass filter and/or high and low pass filter to completely, or partially, suppress unwanted signals.

Instructions executed by control module 417 (also called control logic) can be stored in memory components, encoded in logic circuitry, or a combination of the two. Control module 417, may advantageously comprise control logic or other substrate configuration representing data and instructions, which cause control module 417 to operate in a specific and predefined manner as described herein. Control logic may include, by way of example, components, such as, processes, functions, subroutines, procedures, attributes, class components, task components, object-oriented software components, segments of program code, drivers, firmware, micro-code, circuitry, data, and the like. Control logic may be installed using a computer interface. The computer interface may also be configured to allow a user to vary the control logic, either according to pre-configured variations or customizably. Advantageously, control logic may be installed or varied using remote wireless access.

As disclosed above, microchip 203 comprises a power management module 415 which relays power to control module 417. Power is derived from RF energy harvesting from on-chip coil 413. Power management module 415 also relays power to modulator 419 which is coupled to on-chip coil 413. Microchip 203 further comprises a neural amplifier 425 responsive to electrode(s) 201. Low-noise amplifier 425 is coupled to multiplexer 423 which is further coupled to analog-to-digital converter 421. Output from ADC 421 is coupled to control module 417.

EEG hub device 105 comprises a transceiver 403 module a field-programmable gate array module 405 and a processor 407. The foregoing may advantageously be achieved in a software-defined radio (“SDR”) 401. Hub device 105 may preferably include an amplifier module 411 responsive to SDR 401. Duplexer 409 is interposed between amplifier 411 and SDR 401 and is also coupled to epidermal antenna 103. EEG hub 105 may incorporate transceiver and antenna, such as a Bluetooth® system, to wirelessly communicate with another device, e.g., smartphone. In another embodiment, EEG hub 105 may have a memory to store the EEG data for later retrieval.

Operation of sensor strip 101 will be explained with reference to FIG. 5 which presents a detailed functional schematic of sensor strip 101. In operation, for example, neural activity, including epilepsy activity, can activate one or more electrodes 201a-d which then relay signals 502a-d via leads 207 representing such neural activity to neural amplifiers 425a-d which in turn couple the respective amplified signals 502a-d to 4-to-1 multiplexer 423. Although this exemplary system presents a four-channel configuration with a dedicated amplifier 425 for each electrode 201, it will be appreciated that the sensor strip 101 may be configured with more channels to accommodate more electrodes. Likewise, multiplexer 423 may be substituted to facilitate the routing of signals.

Multiplexer 423 combines amplified analog neural signal 504 from multiple channels, which are then converted to a digitized neural signal 506 by ADC 421. Digitized neural signal is provided as input to control module 417. Control module 417 may be hard encoded with a unique network address 515 so that multiple sensor strips may be in communication with EEG hub device 105. To this end, control module 417 is configured to perform data compression to optimize the transmission and analysis of data. Control module 417 then packetizes digitized neural signals 506 in a shared-medium network protocol frame using shared medium network protocol, for example, a time-division multiple access (TDMA) or a code-division multiple access (CDMA) method with a unique addressing scheme 515, which may be, for example a Gold code. Control module 417 outputs such framed messages as baseband signals 510 which are coupled to modulator 419 which outputs a passband signal 512 to on-chip coil 413 via voltage-controlled switch 509. A non-limiting example of a modulation scheme may be phase-shift keying (“PSK”), in particular, binary PSK (“BPSK”). Passband signal 512 is coupled to antenna 103 as a backscatter data signal 404 where it is imparted to EEG hub device 105 as described in greater detail below.

Microchip 203 can perform spectral filtering to reduce unwanted noise as well as to isolate specific bands which are markers for specific suspected neuropathology. The specific signal processing features of microchip 203 can be programmed using remotely controlled wireless commands depending on the specific clinical case and an individual's neurological condition. Such remote commands may allow for, among other things, dynamic adjustment of signal bandwidth to isolate EEG bands of special interest, setting noise amplitude thresholds, and modulating transmitted data rate. Microchip 203 may include certain on-chip tasks, including, but not limited to, classification components designed to perform epilepsy seizure detection.

The wireless transmission protocol plays a pivotal role in facilitating seamless transmission of neural data from multiple implants which are part of one single RF network. Within this framework, the implants transmit data in a manner that enables EEG hub 105 to communicate with multiple implants concurrently without data interference. Such an autonomous and automatic process eliminates the need for downlink communication, that is sending instructions remotely from the external hub. By adopting this approach, the system can accommodate upwards of a hundred implants with a negligible risk of data collisions in preserving the transmitted data error-free and free from crosstalk interference. Consequently, the aggregate channel capacity can scale to accommodate a minimum of 400 channels (each channel referring to data recorded by an individual electrode on a given sensor strip) thereby surpassing the potential scalability of all other subcutaneous EEG recording devices of the present art.

Power for microchip 203 is harvested from energy imparted by EEG hub device 105 by a transmit signal (FIG. 4: 402) through epidermal antenna 103 through backscatter coupling with on-chip antenna 413. On-chip antenna 413 is coupled to rectifier 503 which provides a direct current to power regulator 505. Control module 417 receives voltage (VDD) 514 from regulator 505, as do oscillator 507 and modulator 419. Oscillator 507 inputs clock signal 508 to control module 417 and modulator. In one embodiment, the EEG hub device 105 provides transmit signal (FIG. 4: 402) in the range of about 915 MHz, for example, for energy transfer and receives a backscatter data signal 404 at about 945 Mhz.

Electrodes 201 are preferably configured to be responsive to impulses from brain activity in the frequency range from about 0.1 Hz to about 100 Hz. Electrodes 201 are preferably about 500 μm in diameter if circular or up to 500 μm×800 μm if using a rectangular geometry, the long dimension lined along the strip with a thickness of up to about 1-3 μm. Multiplexer 423 may include voltage booster circuits to ensure stable switching operation. Further, ADC 421 may be implemented in a preferred embodiment.

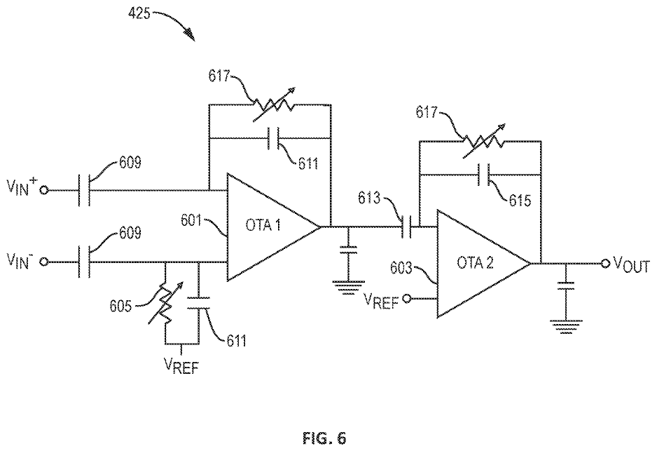

In another embodiment, low-noise neural amplifier 425 may be a two-stage front-end amplifier preferably employing operational transconductance amplifiers (OTAs) to obtain high gain. As illustrated in FIG. 6, the first stage OTA 601 may use pseudo-resistor 605 and feedback capacitor 611 to provide a feedback loop, which also decides the low cutoff frequency. Input capacitor 609 and feedback capacitor 611 determine the gain of the amplifier while the size of input capacitor 609 is constrained due to the limited area of the microchip 203. The second stage OTA 603 provides further amplification and bandpass filtering. Given the limited microchip 203 layout area of no more than 500 μm×800 μm, these capacitance values must be carefully optimized.

Because of the small and unobtrusive size of the thin sensor strip, one or more strips can be implanted into the patient's subcutaneous space through a minimally invasive surgical procedure such as in a doctor's office during a regular visit. The microthread-like implants can be inserted using extractable carriers, such as small size hypodermic needles, requiring only a small initial incision in the skin.

A focused-ion beam (FIB) system, a tool known for its capacity for nanomachine and nanopatterning in a few nanometer resolutions, can be utilized to create microelectrodes 201, 209 with the desired impedance for neural recording. Initially, FIB milling should be performed to remove a localized area of aluminum oxide layer from electrode pads, thereby effectively exposing the underlying aluminum layer. Subsequently, platinum was deposited by FIB-induced deposition. This method provides a way to fabricate neural recording electrodes directly on the chip without resorting to complicated fabrication techniques while parallel processing is not possible.

The system 100 may be configured with features to make it robust against environmental interference and allow the sensor to be used in parallel with other complementary diagnostic medical device techniques such as MRI imaging. The remotely controlled microchip, hence the sensor as a whole, allow for wireless EEG recording under simultaneous neurophysiological excitation such as transcranial magnetic stimulation (TMS). For example, microchip 203 can be disabled in an environment with strong electromagnetic energy.

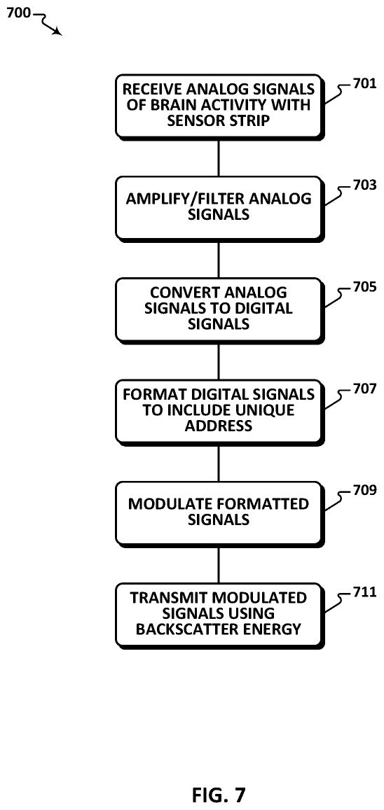

A method 700 performed by system 100 is described with reference to FIG. 7 where at Step 701 a sensor strip 101a-d, and in particular, microchip 203 receives analog signals indicative of brain activity. The analog signals are amplified, and possibly filtered, with amplifier 425 at Step 703. At Step 705, the ADC 421 converts the analog signals to digital signals which are then formatted, e.g., packetized, by the digital signal processor 517 according to a shared medium network communication protocol such as TDMA or CDMA at Step 707. The formatted signals are then modulated (Step 709). Finally, the modulated signals are transmitted at Step 711 using backscatter energy.

As described above and shown in the associated drawings, the present invention comprises a subcutaneous multichannel wireless electroencephalography system. While particular embodiments have been described, it will be understood, however, that any invention appertaining to the system and method described is not limited thereto, since modifications may be made by those skilled in the art, particularly in light of the foregoing teachings. It is, therefore, contemplated by the appended claims to cover any such modifications that incorporate those features or those improvements that embody the spirit and scope of the invention.

1. A sensor device for subcutaneous electroencephalography comprising:
at least one flexible biocompatible polymer sensor strip adapted to be inserted into a cranial subdermal space, said sensor strip comprising:
a plurality of electrodes; and
a microchip responsive to each of said plurality of electrodes, said microchip comprising a wireless transceiver, a low-noise neural amplifier, a unique network address, and RF energy harvesting microcircuits.

2. The sensor device of claim 1, wherein said at least one flexible sensor strip is no more than about 1 mm wide, at least about 5 cm long, and no more than about 300 μm thick.

3. The sensor device of claim 2, wherein said plurality of electrodes is at least four electrodes.

4. The sensor device of claim 3, wherein each electrode of said plurality of electrodes is responsive to brain activity exhibiting frequencies between about 0.1 Hz to about 100 Hz.

5. The sensor device of claim 3, wherein each electrode of said plurality of electrodes is about 500 μm in diameter.

6. The sensor device of claim 1, wherein said microchip comprises a multi-turn on-chip antenna coil for coupling external RF energy to said RF energy harvesting microcircuits.

7. The sensor device of claim 7, wherein said wireless transceiver is configured to transmit signals representative of brain activity using modulated backscatter RF energy.

8. The sensor device of claim 1, wherein said microchip is configured to communicate on a network using a network communication protocol.

9. The sensor device of claim 1 further comprising
an external transceiver disposed on the exterior of the subject's skin near said subcutaneous sensor in secure wireless communication with said wireless transceiver and configured to demodulate said signals.

10. The sensor device of claim 9, further comprising an external, epidermal antenna coupled to said external transceiver such that it is interposed between said external transceiver and said microchip.

11. The sensor device of claim 9, wherein said microchip comprises a planar antenna coil for coupling RF energy from said external transceiver to said RF energy harvesting microcircuits.

12. The sensor device of claim 11, wherein said flexible sensor strip is no more than about 1 mm wide, at least about 5 cm long, and no more than about 300 μm thick.

13. The sensor device of claim 12, wherein each electrode of said plurality of electrodes is responsive to brain activity exhibiting frequencies between about 0.1 Hz to about 100 Hz.

14. The sensor device of claim 13, wherein each electrode of said plurality of electrodes is at least about 500 μm in diameter.

15. The sensor device of claim 14, wherein said at least one sensor is a plurality of sensors.

16. The sensor device of claim 15, wherein the plurality of sensors is up to about 400 sensors.

17. The sensor device of claim 9, wherein said external transceiver collects backscattering signals from said at least one sensor strip using a network communication protocol.

18. The sensor device of claim 9, wherein said external transceiver is configured to be in communication with a wireless communication device.

19. A method for collecting electroencephalography data comprising the steps of:
inserting into a subject's cranial subdermal space, a biocompatible subcutaneous sensor, said sensor comprising:
at least one flexible sensor strip, said at least one flexible sensor strip comprising:
a plurality of electrodes;
a microchip responsive to said plurality of electrodes, said microchip comprising a wireless transceiver configured to transmit signals representative of brain activity using modulated backscatter RF energy, a unique network address, and RF energy harvesting microcircuits; and
placing an external transceiver on the exterior of the subject's skin in close proximity to said sensor, said external transceiver in secure wireless communication with said wireless transceiver and configured to demodulate said signals.

20.-23. (canceled)

24. A method of electroencephalography comprising the steps of:
receiving analog signals representative of brain activity with at least one biocompatible sensor strip inserted into a subject's cranial subdermal space, said sensor strip comprising:
a plurality of electrodes, each said electrode adapted to be responsive to brain activity exhibiting frequencies between about 0.1 Hz to about 100 Hz; and
a microchip responsive to said plurality of electrodes, said microchip comprising an on-chip wireless transceiver configured to transmit signals representative of brain activity using modulated backscatter RF energy, and a unique network address;
converting said signals from analog to digital signals;
wirelessly transmitting said digital signals to an external wireless transceiver located on the exterior of the subject's skin using modulated RF backscatter energy.

25.-28. (canceled)
